(19) United States
(12) Patent Application Publication
(10) Pub. No.: US 2023/0218517 A1
(43) Pub. Date: Jul. 13, 2023

(54) POWDER FORMULATION FOR INGREDIENT DISPERSION

(71) Applicant: TabBrands Inc., Laguna Beach, CA (US)

(72) Inventors: Michael Gulyas, Laguna Beach, CA (US); Dean Harris, Norwalk, CT (US)

(21) Appl. No.: 17/570,957

(22) Filed: Jan. 7, 2022

(51) Int. Cl. A61K 9/00; A23L 2/40

(52) U.S. Cl. CPC A61K 9/0095; A23L 2/40

(57) ABSTRACT

The present invention is a powder formulation designed to function as an insoluble ingredient disperser. The invention demonstrates the ability to improve water distribution, uniformity, and the texture of final preparations. Its composition also provides faster hydration and dispersion of ingredients in primed mixtures and other existing ingredients. A delivery system assists with the hydration of insoluble, oil-based, or poorly dispersible ingredients and create a variety of emulsification solutions for easier and faster processing. The present invention also increases the expansion of extruded products. Varying amounts of the powder formulation can be added as needed to further improve the dispersion and uniformity of final preparations.

### FIELD OF THE INVENTION

The present invention pertains to a powder formulation that assists the dispersion of insoluble, oil-based, or poorly dispersible ingredients in liquids. More particularly, its composition improves uniformity, hydration, extrusion, texture, and processing of final preparations that include, but are not limited to, primed mixtures, additives for various interventions in liquid, tablet, or capsule form, and cosmetics.

### BACKGROUND OF THE INVENTION

The dispersion of liquid formulations is often dependent upon the particles with which they are comprised. For many products, there is a liquid stage where the behavior of the particles dispersed in the solution can determine the ultimate consistency of the final liquid product. Challenges arise due to the limited understanding of the molecular processes that control the final states of the particles. Additional challenges include whether the particles are insoluble, are in the form of a formulated product, are poorly dispersible ingredients, are compounds that don't hydrate, or are oil-based compounds.

Powdered ingredients are among the most widely used compounds in the manufacturing of supplements and other consumables or topical products. More specifically, the use of powder additives and additional functional ingredients such as flavors, nutrients, colorants, and texturizing additives for product preparation has rapidly increased. Although these types of ingredients offer flexibility and consistency for manufactured products, they also present distinct challenges in regards to the incorporation of powder dispersion into the processing stage.

While some forms of powdered or insoluble ingredients are cooperative to dissolution in liquid, other compounds are not as accommodating. Ingredients that are typically difficult to work with include powders used as thickening agents, high-molecular-weight hydrocolloids, starches, proteins, oil-based compounds, and cellulose gums. In general, poorly dispersible ingredients and particularly dry or oil-based compounds resist integration into liquids, making liquid dispersion even more challenging. Accordingly, these types of ingredients pose formidable mixing difficulties for food, beverage, dietary supplement and other product preparation.

Stabilizing proteins in their functional structure is a particularly challenging aspect of product development. Each stage of the manufacturing process as well as storage is influenced by multiple factors that include: shearing, shaking, temperature changes, impurities, protein concentration, solvent interaction, the presence of additives, freezing, and thawing, among others. When proteins are subjected to any of these stressors, degradation can lead to unfolding and aggregation. Protein aggregates decrease efficacy and may stimulate immunogenicity that can be linked to harmful side effects. Stabilizing agents help protect against protein aggregation.

To address these challenges, the present invention comprises of effervescent aids, diluents, stabilizing agents, emulsifiers, and hydration aids that improve ingredient dispersion in liquids. The benefits of enhanced liquid dispersion extend toward a wide variety of formulations or additives for various supplements, interventions (e.g., liquid, tablets, capsules), cosmetics, etc.

### SUMMARY OF THE INVENTION

The present invention is a powder supplement comprised of effervescent aids (e.g., potassium bicarbonate, sodium bicarbonate, citric acid), diluents (e.g., mannitol, sorbitol, xylitol, pearlitol, erythritol), emulsifiers (e.g., sunflower lecithin, locust bean gum, xanthan gum), and hydration aids (e.g., maltose rice syrup, rice dextrin syrup, glycerin, rice bran extract) that enable insoluble, oil-based, or poorly dispersible ingredients to completely break apart and dissolve when they are added to any liquid, including carbonated and non-carbonated liquids.

A carbonated liquid should induce more rapid carbonation and dissolution of active ingredients due to the inherent CO₂ levels of the carbonated liquid. Furthermore, the effervescent components in the powder formulation along with the floating delivery system allow for convenient administration in carbonated and non-carbonated liquids. The specific composition of the powder formulation is designed to facilitate the quicker and more complete dispersion of active ingredients that are insoluble, oil-based, or poorly dispersible into a liquid. In particular, the formulation functions as an ingredient disperser that improves uniformity, texture, and water distribution of active ingredients in final preparations. Its composition also promotes faster hydration of ingredients in primed mixtures and other existing ingredients. Accordingly, the delivery system assists with the hydration of insoluble, oil-based, or poorly dispersible ingredients to create a variety of emulsification solutions for easier and more rapid processing.

In one embodiment, the powder formulation is comprised of the following components: potassium and/or sodium bicarbonate, citric acid; mannitol and/or sorbitol, xylitol, pearlitol, erythritol; maltose rice syrup and/or, rice dextrin syrup, glycerin, rice bran extract; sunflower lecithin and/or locust bean gum, xanthan gum.

Various components of embodiments of the formulation have the following concentrations expressed in approximate:
Potassium bicarbonate (1-50 mg)
Sodium bicarbonate (1-40 mg)
Citric acid (1-40 mg)
Mannitol (1-25 mg)
Sorbitol (1-25 mg)
Xylitol (1-25 mg)
Pearlitol (1-25 mg)
Erythritol (1-25 mg)
Rice bran extract (1-20 mg)
Maltose rice syrup (1-20 mg)
Rice dextrin syrup (1-20 mg)
Glycerin (1-20 mg)
Sunflower lecithin (1-15 mg)
Locust bean gum (1-15 mg)
Xanthan gum (1-15 mg)

Various embodiments of the composition of the present invention include at least one of the components set forth above.

### DETAILED DESCRIPTION OF THE INVENTION

The present invention is a powder formulation designed to function as an ingredient disperser. The invention demonstrates the ability to improve water distribution, uniformity, and the texture of final preparations. Its composition also provides faster hydration and dispersion of ingredients in primed mixtures and other existing ingredients. The delivery system assists with the hydration of insoluble, oil-based, or poorly dispersible ingredients and create a variety of emulsification solutions for easier and faster processing. The present invention also increases the expansion of extruded products. Varying amounts of the powder formulation can be added as needed to further improve the dispersion and uniformity of final preparations. The formulation is comprised of effervescent aids (e.g., potassium bicarbonate, sodium bicarbonate, citric acid), diluents (e.g., mannitol, sorbitol, xylitol, pearlitol, erythritol), emulsifiers (e.g., sunflower lecithin, locust bean gum, xanthan gum), and hydration aids (e.g., maltose rice syrup, rice dextrin syrup, glycerin, rice bran extract).

The active ingredients in the present invention are subjected to a micro jet mill process that yields ingredients of 1.5-2 micron size. A mixture of several ingredients is then oscillated together in a tubular mixer in order to achieve an evenly distributed blend. Certain ingredients in the powder formulation are slightly larger in particle size, and micro jet mill processing creates a consistent and uniform powder blend of an optimal micron size. The micro jet mill process and use of tubular mixer yield specific micron sizes for each ingredient in the powder formulation. When this method is applied to the powder formulation blend, a water-soluble and uniformly dispersed solution is achieved. The powder formulation is produced in controlled environmental conditions through various methods that include: direct compression, wet granulation, fluid-bed granulation, and fusion method. As the present invention is sensitive to temperature and moisture, modest temperatures (e.g., 25° C.) and a relative humidity of approximately 25% or less must be maintained during production to prevent granulation or adhesion of the powder formulation to the machinery due to moisture absorption.

For this invention, effervescence is accomplished through the incorporation of potassium bicarbonate or sodium carbonate (or any other carbonate/bicarbonate consisting of alkali metals) and citric acid (or tartaric acid, malic acid, or any other acid anhydride) in the composition of the powder formulation. These ingredients react with water to create an acid-base reaction that releases CO₂ and aids in dispersion of the active ingredients. The CO₂ that is produced through an effervescence reaction increases the penetration of the active ingredient, including insoluble, oil-based, or poorly dispersible compounds, into the paracellular pathway and subsequently increases ingredient absorption. Effervescent refers to the mechanism by which the combination of active ingredients (e.g., insoluble, oil-based, or poorly dispersible compounds) and this powder formulation react with a liquid to induce the release of gas.

Moreover, the powder formulation incorporates diluents in the present invention. Diluents are substances that aid in the breakdown of the large, insoluble, oil-based, or poorly dispersible ingredients into a liquid. In particular, diluents aid the breakup of a compacted mass when it is put into a liquid environment. Accordingly, diluents promote moisture penetration and dispersion of the powder. Naturally-sourced diluents include starches, partially pre-gelatinized starches, and hydrolyzed starches. Common diluents for supplement or product formulation include lactose monohydrate and anhydrous lactose as well as sugar alcohols such as mannitol, sorbitol, pearlitol, erythritol, and xylitol.

Certain diluents (e.g., pearlitol, erythritol, sorbitol, mannitol) also function as stabilizing agents that address the complexity and natural propensity of proteins to aggregate, which is a common challenge during the product preparation stage. Stabilizing agents enhance protein protection against humidity, demonstrate high water solubility, and help shield water insoluble materials, thereby regulating protein aggregation. As primed mixtures or other existing ingredients may contain poorly dispersible compounds, the powder formulation incorporates one or multiple diluents that assist the breakdown of such compounds once they reach a liquid.

In addition, the present invention incorporates one or multiple emulsifiers that promote the dispersion of large, insoluble, oil-based, or poorly dispersible ingredients in liquid. Emulsifier molecules have a hydrophilic end (water-attracting) and hydrophobic end (water-repelling). When added to a liquid, the emulsifier surrounds the poorly dispersible droplet with the hydrophobic end extended towards the active ingredient (e.g., insoluble or oil-based compound) and the hydrophilic end faces the water or liquid. This reduces the interfacial tension between the active ingredient and the liquid (e.g., water), thusly stabilizing the droplets and preventing them from coalescing. Common emulsifiers include sunflower lecithin, locust bean gum, xanthan gum, rice bran extract, sodium mono-and di-glycerols, ammonium phosphatide, and stearoyl lactylate. Emulsifiers form physical barriers that prevent compounds within a liquid mixture from coalescing. Additionally, emulsifiers such as sunflower lecithin, xanthan gum, and locust bean gum enhance the dispersion/hydration of active ingredients in hot or cold liquids. There are also many different grades of gums readily available. Although emulsifier (gum) powders have a strong tendency to form agglomerates when exposed to water, premixing emulsifiers with other powdered ingredients such as diluents reduce agglomerate formation by separating the particles. These types of emulsifiers are favorable for a variety of preparations (e.g., liquids, tablets, capsules) as their polysaccharide components generally form ordered structures in aqueous environments that support particle uniformity, a smooth texture, faster hydration, and complete dispersion.

The powder formulation also contains hydration and extrusion aids (e.g., maltose rice syrup, rice dextrin syrup, glycerin, rice bran extract). Hydration aids help direct moisture into insoluble, oil-based, or poor dispersible ingredients quickly and more uniformly. The hydration and extrusion properties of agents such as glycerin and rice bran extract improve uniformity, lower bulk density, reduce breakage, and increase water distribution to the active ingredients. These properties also increase extrusion output and the expansion of extruded ingredients. Accordingly, final preparations demonstrate smoother surfaces, texture, and visibility (clearness), as well as improved extrusion performance.

In regards to the delivery system, the dispersion of the powder formulation within liquid may take approximately 5-20 seconds or longer to fully disperse within if the final preparation contains large, insoluble, oil-based, or poorly dispersible ingredients (e.g., starches, hydrocolloids, proteins, cellulose gums). The powder formulation is designed to break apart and dissolve in 3 to 4 ounces of water relatively quickly, for example, after being added or stirred in a container of liquid for a short period of time ranging from several seconds to a few minutes.

Furthermore, the delivery system is a low-density formulation that stays afloat over the gastric fluid and remains buoyant in the stomach without adversely affecting the rate of gastric emptying. This system yields better absorption and increased bioavailability. The buoyancy of the delivery system is assisted by low density agents and compounds such as sodium bicarbonate, citric acid, tartaric acid, or other metal carbonates or alkali metal bicarbonates. These agents are present in powder form and are capable of releasing CO₂ when they are formulated with active ingredients and other auxiliaries to provide granules without the premature evolution of CO₂. The delivery system utilizes effervescent and dispersion-enhancing reactions when the powder formulation comes in contact with the gastric fluid. This is due to the CO₂ gas that is generated from the gastrointestinal system when it is exposed to liquid as water or other liquids act as catalyzing agents that increase the rate of the reaction. The floating delivery system is designed to yield a powder formulation density that is lower than that of gastric fluid in order for the active ingredients to remain buoyant for extended time periods without affecting the gastric system's emptying rate. In a non-effervescent floating delivery system, the active ingredients swell in the gastric fluid while maintaining relative stability of their shape and a bulk density that is less than the density of the gastric fluid. This augments the floating process of the active ingredient serving forms.

The present invention incorporates an effervescent floating delivery system that is based on effervescent components (e.g., potassium bicarbonate, sodium bicarbonate, citric acid) which liberates CO₂ due to the acidic properties of the gastric fluid. The liberated CO₂ gas becomes entrapped in the floating layer formed by the hydrocolloids (e.g., sunflower lecithin, locust bean gum, xanthan gum) and this reaction leads to an upward motion of the active ingredients that also maintains its buoyancy.

Additionally, the floating delivery system of this invention might offer faster biological effects than other insoluble ingredient dispersers. A non-effervescent solution may also have a bad taste and high viscosity that might increase its intolerability in the digestive tract. Furthermore, the composition of this powder formulation increases the ease of administration and improves the absorption of the active ingredients through the previous dissolution in a buffered liquid medium. In addition, a floating delivery system can buffer the aqueous solution of the active ingredients, thereby increasing the stomach pH which prevents the inactivation or degradation of the active ingredients. The buffering effect through carbonation may also induce quicker emptying of the stomach, usually within 20-30 minutes into the small intestine, as this promotes maximum absorption of active ingredients.

Accordingly, the components in the present invention are advantageous as active ingredients (e.g., large proteins, insoluble powders, starches, etc.) that are combined with this ingredient dispersing powder formulation is already be in solution at the time they are consumed, which makes the absorption faster and more complete in comparison to other formulations. The floating delivery system enables the active ingredients to fully dissolve in a buffered solution and this reduces localized contact in the upper gastrointestinal tract. This results in less irritation and greater tolerability of the final preparation. This buffering mechanism also prevents gastric fluids from interacting with active ingredients themselves, which is a major causative factor for esophageal and intestinal upset. Moreover, this floating delivery system produces a homogenous final preparation that may have better consumer appeal than viscous, non-effervescent solutions. Additionally, this delivery system helps the active ingredients retain their palatability after lengthy storage times. Excellent stability is also inherent to a powder formulation with a floating delivery system, particularly surpassing that of non-homogenous liquid solutions.

Active ingredients delivered through floating delivery systems also have reproducible and predictable bio-kinetic profiles that are more consistent than capsules or tablets, as effervescent components assist the therapeutic profiles of the active ingredients. The combination of effervescent, diluent, stabilizing, emulsifying, and hydrating components also facilitate the solubilization of ingredients that generally demonstrate a poorly dispersible nature. A floating delivery system enhances the penetration of a broad range of active ingredients that vary in size, structure, and other physiological properties. In addition, the present powder formulation can be used for programmed product administration in remote areas and this type of formulation addresses the issue of physicochemical stability and the high cost of transporting capsules, tablets, or viscous syrups. Therefore, a powder formulation is a realistic solution to enhanced liquid dispersion, easy administration, maximum absorption, sustained active ingredient stability in the gastrointestinal tract, and a low incidence of stomach upset, in addition to other advantages.

The concept of dispersion-enhancing powder is mainly based on the mixture of ingredients such that the active ingredients remain embedded in the delivery system in which the active ingredients are released, without the disintegration of the powder formulation. The powder formulation may also be used as a sustain release dosage form to overcome problems that are associated with conventional dosage forms. This also reduces fluctuations of active ingredient(s) concentration and enhances the bioavailability of components in the final preparations. Thusly, the present intervention demonstrates an optimal floating delivery system in comparison to current delivery systems for insoluble, oil-based, or poorly dispersible ingredients typically found in effervescent tablets, powders, viscous solutions, and topicals.

Additional applications include, but are not limited to the preparation of a tablet containing the insoluble ingredient dispersion powder for the purpose of targeted delivery, floating delivery, or extended-release. Similarly, the ingredient dispersion powder may be prepared in a capsule form to enhance the delivery of plant-based or oil-based particles. Tablet and/or capsule preparations that contain ingredient dispersers, particularly emulsifiers such as locust bean gum, lecithin, or xanthan gum, are favorable for sustained release or controlled released formulations. Furthermore, tablets/capsules that are prepared with emulsifiers facilitate gastrointestinal specific or colonic specific delivery, with emulsifiers such as xanthan gum or locust beam gum functioning as the primary carriers and excipients.

The benefits of enhanced liquid dispersion that this invention offers extends to a wide variety of formulations or additives for various supplements, interventions (e.g., liquids, tablets, capsules), cosmetics, etc. In particular, improved uniformity, hydration, stabilization, extrusion, texture, and processing of active ingredients in final preparations can be achieved through the use of this powder formulation that functions as an insoluble ingredient disperser. These properties can help deliver highly bioavailable compounds through oral consumption and topical applications. This single invention addresses the challenges involved with improving the liquid dispersion of poorly dispersible ingredients that include protein stabilizing, oil emulsion, hydration, and extrusion performance.

From the above description, it is clear that the inventive concept(s) disclosed herein is well adapted to carry out the objects and to attain the advantages mentioned herein as well as those inherent in the inventive concept disclosed herein. While exemplary embodiments of the inventive concept disclosed herein have been described for purposes of this disclosure, it will be understood that numerous changes may be made which will readily suggest themselves to those skilled in the art and which are accomplished without departing from the scope of the inventive concept disclosed herein and defined by the appended claims.

What is claimed:

1. A powder formulation for dispersing insoluble ingredients in a liquid, comprising:
at least one effervescent agent that reacts with liquid to create an acid-base reaction that releases CO₂;
at least one diluent to aid in the breakdown of large, insoluble, or poorly dispersible ingredients;
at least one stabilizing agent for addressing the complexity and natural propensity of proteins to aggregate;
at least one emulsifier for stabilizing active ingredient droplets and prevents them from coalescing; and
at least one hydration aid for directing moisture into insoluble, oil-based, or poorly dispersible ingredients quickly and more uniformly.

2. The powder formulation of claim 1 wherein the powder formulation is a low-density formulation that promotes ingredient dispersion by breaking apart and dissolving when added to any liquid, including carbonated and non-carbonated liquids.

3. The powder formulation of claim 1 wherein the powder formulation improves uniformity, texture, water distribution of active ingredients in final preparations.

4. The powder formulation of claim 1, wherein the powder formulation is combined with active ingredients to create a reaction with liquid that induces the release of gas.

5. The powder formulation of claim 4 wherein the active ingredients are insoluble, oil-based, or poorly dispersible compounds.

6. The powder formulation of claim 1 wherein the powder formulation is designed to break apart and dissolve in 3 to 4 ounces of water relatively quickly, after being added or stirred into the liquid for a short time period ranging from several seconds to a few minutes.

7. The powder formulation of claim 1 wherein the powder formulation is designed to increase the ease of administration and improve the absorption of active ingredients through a previous dissolution in a buffered liquid medium.

8. The powder formulation of claim 1 wherein the powder formulation is designed to produce uniform dispersion and reduce fluctuations of active ingredient(s) concentration to enhance the bioavailability of components in final preparations.

9. The powder formulation of claim 1 wherein the powder formulation is added as needed to further improve the dispersion and uniformity of final preparations.

10. The powder formulation of claim 1, further comprising:
a floating delivery system that assists the hydration of insoluble, oil-based, or poorly dispersible ingredients to create a variety of emulsification solutions for easier and more rapid processing.

11. The powder formulation of claim 10 wherein the floating delivery system promotes liquid dispersion of approximately 5-20 seconds or longer to fully disperse ingredients within a final preparation.

12. The powder formulation of claim 10 wherein the floating delivery system has a low-density composition that stays afloat over the gastric fluid and remains buoyant in the stomach without adversely affecting the rate of gastric emptying.

13. The powder formulation of claim 10 wherein the floating delivery system liberates CO₂ due to the acidic properties of the gastric fluid, where liberated CO₂ gas becomes entrapped in the floating layer formed by the hydrocolloids to create a reaction that leads to an upward motion of the active ingredients that also maintains its buoyancy.

14. The powder formulation of claim 10 wherein the floating delivery system utilizes effervescent and dispersion-enhancing reactions when the powder formulation comes in contact with the gastric fluid.

15. The powder formulation of claim 10 wherein the floating delivery system offers faster biological effects than other insoluble ingredient dispersers.

16. The powder formulation of claim 10 wherein the floating delivery system produces a homogenous final preparation having better consumer appeal than viscous, non-effervescent solutions.

17. The powder formulation of claim 10 wherein the floating delivery system helps active ingredients retain their palatability after lengthy storage times.

18. The powder formulation of claim 10 wherein the floating delivery system enhances the penetration of a broad range of active ingredients that vary in size, structure, and other physiological properties.

19. The powder formulation of claim 1 wherein the powder formulation is used for the preparation of a tablet containing the insoluble ingredient dispersion powder for targeted delivery, floating delivery, or extended-release purposes.

20. The powder formulation of claim 1 wherein the powder formulation is used for the preparation of a capsule containing the insoluble ingredient dispersion powder to enhance the delivery of plant-based or oil-based particles.

21. The powder formulation of claim 1 wherein the powder formulation is used for the preparation of tablet and/or capsule preparations for sustained release, controlled release, targeted delivery, or system specific delivery of active ingredients.

22. The powder formulation of claim 1 wherein the powder formulation is for the preparation of tablet and/or capsule preparations with emulsifiers that function as the primary carriers and excipients.

23. The powder formulation of claim 1 wherein the powder formulation is produced through a micro jet mill process and a tubular mixer to yield specific micron sizes for each ingredient in the powder formulation for a water-soluble and uniformly dispersed solution.
